(19) United States
(12) Patent Application Publication
(10) Pub. No.: US 2023/0240870 A1
(43) Pub. Date: Aug. 3, 2023

(54) Launcher for Introduction of A Medical Device

(71) Applicants: Keisuke Suzuki, Rancho Palos Verdes, CA (US); Matthew Joseph Fairshter, Pflugerville, TX (US)

(72) Inventors: Keisuke Suzuki, Rancho Palos Verdes, CA (US); Matthew Joseph Fairshter, Pflugerville, TX (US)

(21) Appl. No.: 17/589,817

(22) Filed: Jan. 31, 2022

(51) Int. Cl. A61F 2/95; A61F 2/86; A61M 16/04

(52) U.S. Cl. CPC A61F 2/95; A61F 2/86; A61M 16/0463; A61M 16/049; A61F 2210/0014; A61M 2205/0266

(57) ABSTRACT

A launcher for placement and introduction of a medical device into a body lumen is provided. In one embodiment, the medical device is a nested cannula which is pre-designed and fabricated for access to a lumen within a patient body. The launcher comprises a stent having a plurality of guidewires, the guidewires extending from the stent to an external access point of the patient. The guidewires are positioned within a guidetube and disposed within a plurality of channels therein. In some embodiments, a bronchial access assembly comprising a mouthpiece and a J-tube for access to the trachea is also provided. In another embodiment, a stent for introducing a nested cannula is provided. The stent is comprised of a shape memory alloy or other material designed to be deployed within a body lumen, and plurality of guidewires affixed to the stent. In another embodiment, a method for introducing a nested cannula into a body lumen is provided.

### BACKGROUND

Access to passages within the body, in particular, the bronchi, the air passages beyond the trachea, is often necessary for diagnosis or treatment of the lesions or other abnormalities in the bronchi. Diagnosis and treatment of the lesion or other abnormality can include evaluating an abnormality seen on chest X-ray or CT scan, diagnosing a lung infection or other lung disease; investigating the cause of unexplained fluid collection in the lung; and evaluating whether a lung mass is malignant (cancerous) or benign.

Referring now to FIG. 1, a diagram of the trachea and bronchus is shown. As shown in FIG. 1, the trachea divides into right and left main bronchi at the corina, which in turn form lobar, segmental, and intrasegmental bronchi. Access to the bronchi is complicated due to the narrow pathways, often with sharp curvature, and delicate tissue of the bronchi.

Navigation devices, such as bronchoscopes, have difficulty reaching certain areas of the bronchi due to their size and lack of dexterity. A typical bronchoscope can only reach the largest airways of the lung, leaving large portions of the lung unreachable with this technology.

In traditional surgical techniques, catheters and guidewires have been used to reach deep within the body by following vessels. However, these devices ability to maneuver through challenging turns limit the applicability of the device for areas of the body with complex pathways, or which have sharp and multiple curvatures, and non-elastic tissue wall. In the lung, catheters and guidewires are difficult to control at the distal end to reach specific branches of the lung. In particular, the complex pathways of the lung, and particularly the upper lobes, are difficult to access due to the sharp turn required for access through the trachea and principal bronchus. Further, when these devices are inserted into the pathways of the lung, tissue damage throughout the path traveled to a target can occur as the device is inserted and multiple manipulations are used to traverse the complex pathways.

Devices and methods have been developed for accessing the bronchi, such as Application Nos.: US 2011/0201887; and US 2014/0371532 which describe nested cannulas, referring to a device having a series of nested, length-wise interlocking tubes, to reach a target location within a particular anatomical region of a patient. The nested cannula is positioned within the patient by the sequential deployment of the nested tubes. U.S. Pat. Nos. 8,535,336; US 9,387,047; and US 9,895,163; and Application Nos. WO 2010/076674; and WO/2010113053 describe cannula configuration systems incorporating a customized tool that is created for a specific patient based on a pre-acquired three dimensional (3D) image, and identification of a target location for launching the nested cannula. Typically, a 3D image of the particular anatomical region of an individual patient is used to custom configure and dimension a series of nested tubes having a pre-defined curvature, diameter, and length. The path of the nested tubes is pre-determined and precisely defined with a target location for the launch point and end point at the distal tip of the nested cannula. The nested cannula are then custom fabricated, with respect to the curvature, diameter, and length, and orientation within the patient, which matches the individual anatomy of the patient.

Although the above described nested cannula devices and configuration systems are can be designed and fabricated for deployment within the lung, effective deployment requires precise positioning, within approximately 2 mm. Precise positioning is required at the launch point, without twisting for proper cannula placement and insertion with minimal tissue damage. However, precise placement is difficult, as is proper initial orientation of the device, without which the device does not properly deploy. Accordingly, these deployment challenges have limited the use of this technology.

In addition, minimally invasive techniques for diagnosis and treatment of lesions or other abnormalities in the bronchi are preferable for patient care. Procedures that can be accomplished without general anesthesia are highly preferable due to the complications associated with general anesthesia. A needle or transbronchial lung biopsy, although performed under light sedation and/or local anesthesia can have serious complications including pneumothorax, bleeding in the lung, and infection.

Therefore, an improved device and method for access to passages within a patient's body, in particular, a patient's lung passageway, and more particularly the passageways beyond the corina, is needed. For optimal functionality, the device and methods should have the capability to be deployed without the use of general anesthesia, and on an outpatient basis under light or twilight sedation in a physician office setting.

### SUMMARY

According to the present disclosure, a device, system, and methods are provided for placement and introduction of a medical device in a body lumen. In one embodiment, a launcher composed of a positioner and guidetube is provided. Systems and methods for setting the positioner stent in the body and bronchi, in particular, as a platform for the launch of a nested cannula into a patient's bronchi are also provided. According to one embodiment, the nested cannula is pre-designed and fabricated for access to a pre-determined position within a patient body. The positioner comprises a stent, preferably which is self-expanding, having a plurality of guidewires attached to an inner surface of the stent. The guidewires perform as rails for the guidetube to be advanced to a position at the distal end of the stent. The orientation of the stent and guidetube, meaning position within the patient body, is significant so as to have the correct position to launch other medical devices, such as a nested cannula. The stent is preferably comprised of a shape memory alloy or other material designed to be deployed within a body lumen, and plurality of guidewires affixed to the stent from the proximal to the distal end of the stent.

The positioner is inserted into a patient body with the guidewires extending from the stent to an external access point of the patient, the mouth or the nose. The guidetube is advanced into position inside the stent by advancing the guidetube along the guidewires within their respective channels within the guidetube.

According to another embodiment, a system and method for placing the stent and the guidetube within a patient body are also provided. According to this embodiment, a bronchial access assembly comprising a mouthpiece and a J-tube is provided. The J-tube eases the advancement of the guidetube through the acute bent from the mouth/nose to the trachea, while keeping the orientation of the guidewires the same. In another embodiment, a method for introducing a nested cannula into a body lumen is provided.

The launcher, system, and methods according to the disclosure are minimally invasive and diagnosis and treatment of a medical condition within the bronchi can be accomplished without general anesthesia and on an outpatient basis under light or twilight sedation. The launcher, system, and methods allow for precise access to a patient's lung passageways, particularly the passageways below the corina. The launcher is retrievable after use where the guidetube can be withdrawn. The positioner stent may be retrieved by retracting the stent into an outer sheath. The guidewires facilitate such retrieval by directing the stent back into the sheath. The launcher is a temporary device which fixes a position within the body, particularly the bronchi, for a nested cannula or other device to be advanced from as a designated original point in the body.

Accordingly, the present disclosure addresses problematic issues relating to access to the body, bronchi, and the air passages beyond the trachea, for diagnosis or treatment of the lesions or other abnormalities in the bronchi.

### FIGURES

These and other features, aspects and advantages of the present disclosure will become better understood from the following description, appended claims, and accompanying figures where like numbers reference like elements and where:

FIG. 1 is an illustration showing the trachea and major lobes of the human bronchi;

FIG. 2A is a side view of the stent and guiderails, according to one embodiment;

FIG. 2B is a side view of the stent shown in FIG. 2A with a guidetube fully inserted into the stent and the guiderails positioned within the guidetube;

FIG. 3A is a side view of the launcher, with the guiderails extended through the guidetube, and the guidetube partially inserted into the stent;

FIG. 3B is a side cut-away view of the launcher shown in FIG. 3A;

FIG. 4A is a bottom view of the distal end of the stent shown in FIG. 2, having rails attached at the distal end of the stent for connection of the guiderails to the stent, according to one embodiment;

FIG. 4B is a bottom view of the distal end of the stent shown in FIG. 2, with the guiderails attached to the distal end the stent, according to another embodiment;

FIG. 4C is a bottom view of the distal end of the stent shown in FIG. 2, having rails attached at the distal end of the stent, and also showing the guidetube inserted into the stent, according to one embodiment;

FIG. 4D is a bottom view of the distal end of the stent shown in FIG. 2, with the guiderails attached to the distal end of the stent, according to another embodiment;

FIG. 5A is a top perspective view of a guidetube, showing three optional entry/exit ports, according to another embodiment;

FIG. 5B is a top view of a guidetube, showing two optional ports, according to another embodiment;

FIG. 5C is a side view of a guidetube, showing an optional side port and two entry/exit ports, according to another embodiment;

FIG. 6A is a side view showing the mouthpiece and J tube according to another embodiment of the disclosure;

FIG. 6B is a side cut away view of a patient body showing the mouthpiece, J-tube, and stent inserted into the patient body, according to another embodiment;

FIGS. 7A is a side cut away view of a patient bronchi showing the stent and sleeve inserted into the patient body, according to another embodiment;

FIG. 7B is a side cut away view of a patient bronchi showing the stent and inserted into the patient body, after deployment;

FIG. 7C is a side cut away view of a patient bronchi showing the stent and guidetube after insertion; and

FIG. 7D is a side cut away view of a patient bronchi showing the stent, guidetube, and nested cannula after insertion into the bronchi.

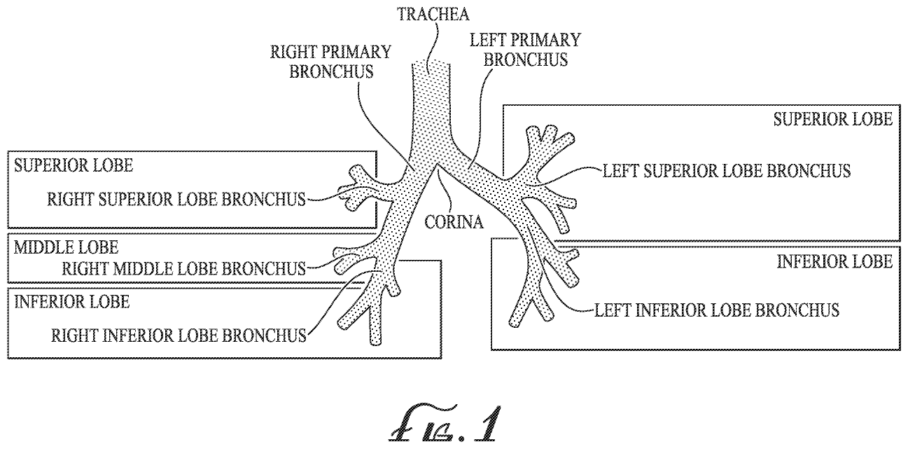

### DESCRIPTION

According to the present disclosure, a launcher 100 for introducing a medical device into a patient body is provided. The launcher 100 comprises a stent with guiderails affixed to the distal end of the stent and a guidetube. The guiderails extend from the distal end of the stent to a position outside the patient's body, such as the mouth or nose. The guidetube is positioned in the interior of the stent, terminating at the distal end of the stent. The guidewires are positioned within channels contained within the guidetube and direct the guidetube into a precise position within the stent. The interior of the guidetube is configured to accept a medical device, such as a nested cannula, in a known orientation within the patient's body. The launcher 100 then acts as a platform for the launch of the nested cannula, as known in the art, into the patient's bronchi. In addition, systems and methods are also described herein for positioning the launcher within the patient's body with precise positioning for the launch point of other medical devices, such as the nested cannula, referred to herein.

Accordingly, the launcher, systems, and methods described herein can be incorporated for use with known tools that create patient based three dimensional (3D) images and can identify the launcher position within a patient's body as the a target location for launching a nested cannula, or other devices within the patient's body. As the path of the nested cannula tubes is pre-determined and precisely defined with the known target location of the launch point, provided by the launcher described herein, the custom fabricated nested cannula can be custom fabricated and effectively deployed within the patient with required precision (e.g., within approximately 2 mm). The precise positioning provided by the launcher allows deployment and insertion of a nested cannula within the bronchi with minimal tissue damage. In addition, the systems and methods described herein for positioning the launcher within a patient's body are minimally invasive and can be performed without general anesthesia, thus minimizing complications.

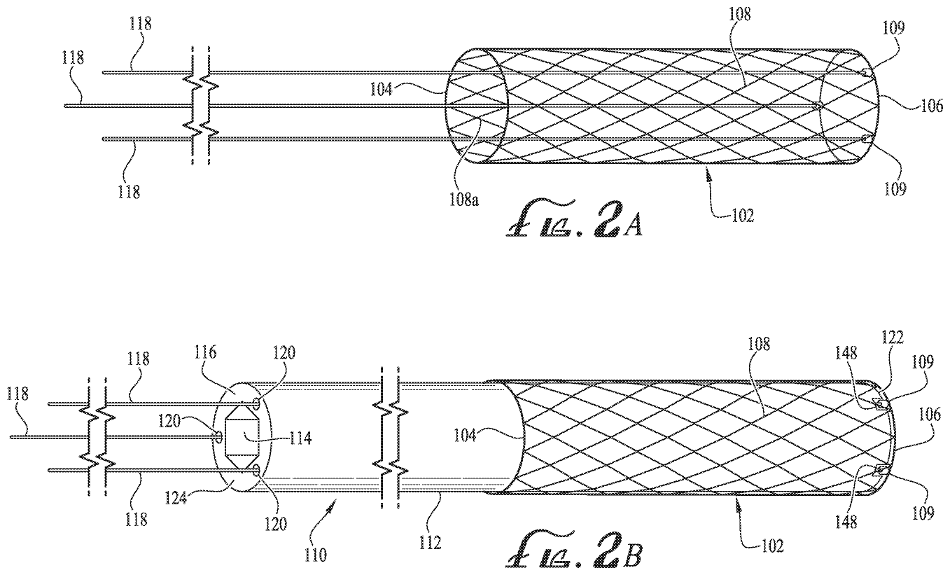

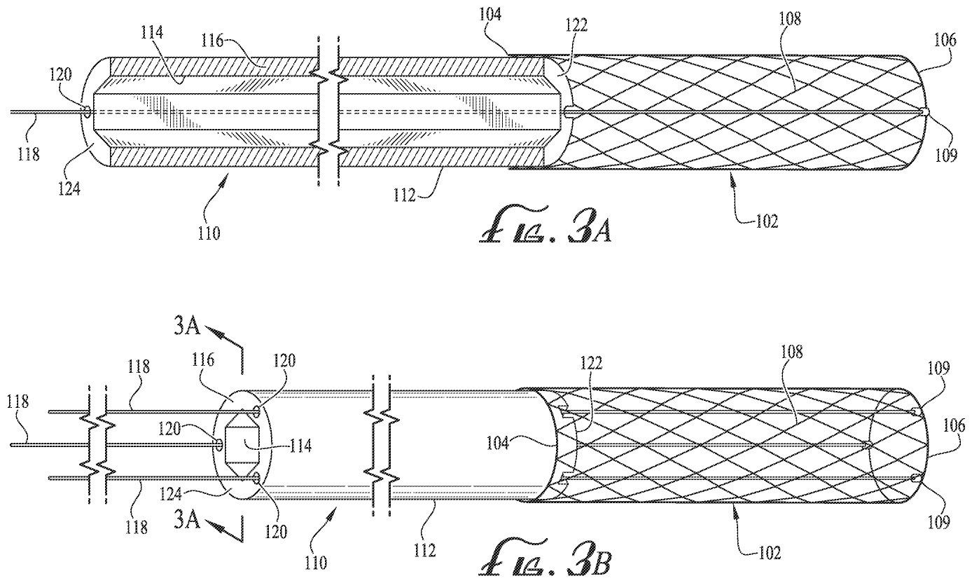

Referring now to FIGS. 2A-5C, various embodiments of the launcher, including stent, guiderails, and guidetube, is shown. Referring now to FIG. 2A, a side view of the stent 102 and guiderails 118 is provided. Referring to FIG. 3A, a side cut-away view of the launcher 100, with the guidetube 110 partially inserted into the stent 102 is shown. Referring to FIG. 3B, a side view of the launcher 100 shown in FIG. 3A is shown. The stent 102, has a proximate end 104, a distal end 106, and a plurality of guidewires 118, affixed to the stent distal end 106. The guidewires 118 are affixed to the stent 102 distal end 106, and extend through the interior of the stent 102, past the stent proximal end 104, and terminate at a length which is sufficient to extend outside a patient body through an external access point of the patient body. Preferably, the stent 102 comprises an airway stent with having a length and an outside diameter sized for an airway passage, ranging from about 8 mm to about 20 mm, preferably about 10 mm, when expanded. Shorter stent 102 lengths are preferable, but depend on the size of the patient and size of the body lumen into which the stent 102 is positioned. The length of the stent 102 will depend on how deep or far the bronchoscope reaches. The stent being mesh, can expand according to the diameter of the bronchus, thus it can be tapered when expanded. When the stent is long, the guidewires may be attached more at the distal end to keep the orientation and have enough strength for the attachment to the stent. For example, a longer stent may be used for distal placement in the bronchus. In this example, the preferred length is from about 25 mm to 30 mm for anchoring the stent 102 in the bronchus. The body 108 of the stent 102 can be a hollow uniform cylindrical shape or expanded at either the distal end 106 or the proximal end 104, depending on where the stent is placed in a patient body. The body 108 of the stent 102 comprises various hollow cylindrical configurations, such as the mesh shown in FIG. 2, and other hollow cylindrical mesh configurations. The stent 102 is comprised of a material suitable for insertion into a body lumen, such as a shape memory alloy, such as Nitinol, or other metallic materials such as tantalum or stainless steel, or polymeric materials, and may be coated or uncoated to facilitate insertion and/or reduce damage to surrounding tissue, such as with silicon, polyurethane, PTFE, and Dacron, for example. In addition, the stent 102 may have a smooth or rough surface, particularly when using a coated stent 102.

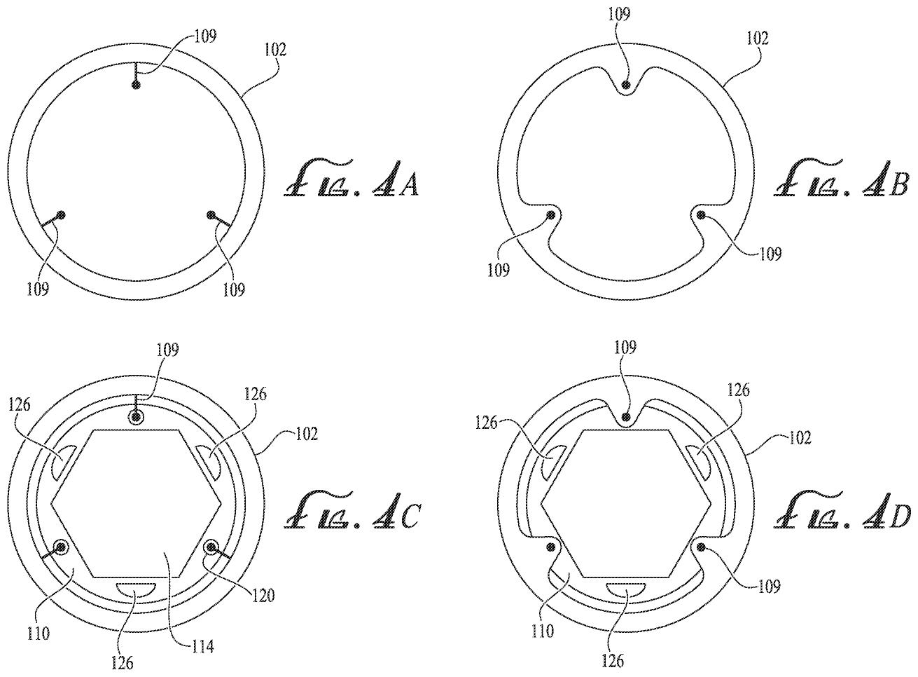

As shown in FIGS. 2A, 3A-3B, and 4A-4D, the guidewires 118 are affixed to the distal end of the stent 102 and extend through the body 108 interior to a length outside the patient's body, such the patient's mouth. The guidewires are affixed at the distal end 106 with a connection member 109 which also acts as a stop for attachment of the guidetube 110. The guidewires 118 are not attached at the proximal end of the stent to allow for insertion of the guidetube 110 into the stent 102, as shown in FIG. 2B. The connection member 109 can have a variety of configurations, such as a rail, extending from the distal end 106 and affixed to the guiderails 118 at a distance, as shown in FIG. 4A; or, as shown in FIG. 4B, a projection extending from the distal end 106 of the stent 102 to affix the guidewires 118 at a distance from the stent body 108. The guidewires 118 are affixed at a distance from the stent body 108, such that guidewires 118 are readily positioned within the guidetube 110 when the guidetube 110 is fully inserted into the stent 108, such as shown in FIGS. 4C and 4D. Other configurations for the connection member 109 are within the scope of the disclosure, such as a lip or flange, that will accommodate attachment of the guiderails 118 at a distance from the stent body 118 such that the guidetube 110 can be inserted into the stent body 108 with a known position and orientation.

Preferably, as shown in FIG. 2A, the stent 102 has three guidewires 118, having a diameter of from about 0.2 mm to about 0.5 mm, and a length of from about 1 m to about 2 m. Preferably, the guidewires 118 are longer than double the length of a bronchoscope. The guidewires 118 may be of the same or different material as the stent 102. Preferably, the guidewires 118 are made of nitinol or stainless steel. The guidewires 118 are affixed to the stent 102 such as by welding, for example, laser welding the guidewires 118 to the connection member 109 on an inner surface of the stent 102. However, the stent 102, including guidetube 110 and guidewires 118 can be of varying size, depending on the positioning within the patient body and size of the patient.

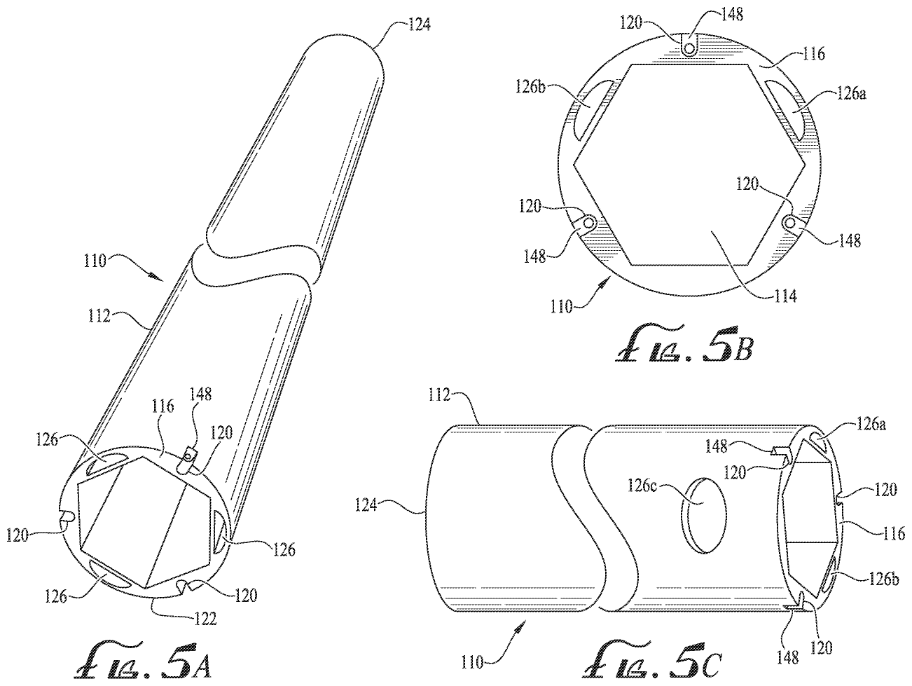

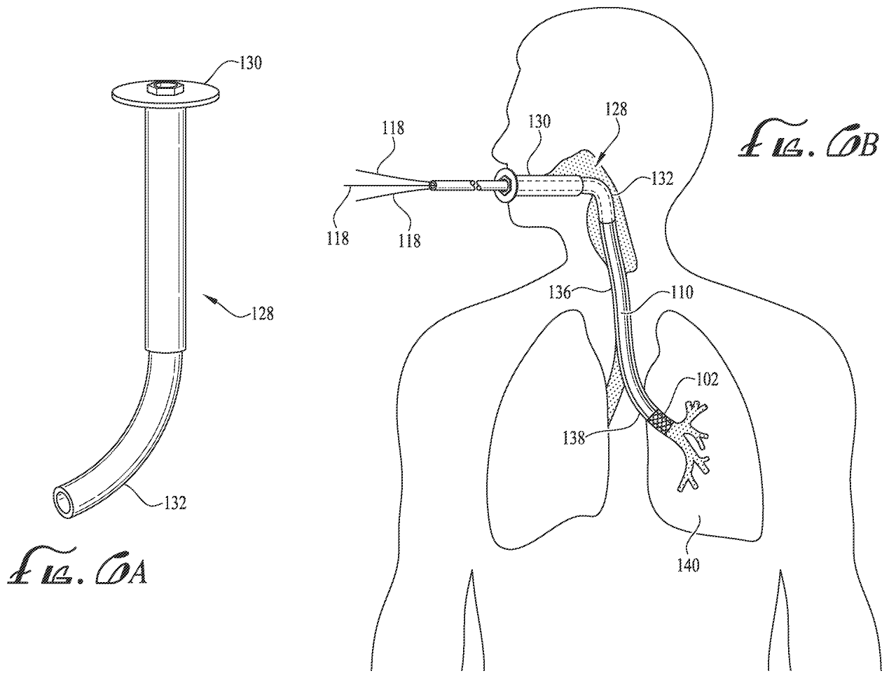

Referring now to FIGS. 3A-3B and 5A-5C, the guidetube 110 comprises a plurality of channels 120 disposed within the tube wall 116. The channels 120 are preferably oriented 120 degrees apart. The guidewires 118 are removably contained within the plurality of channels 120, and the plurality of channels 120 are oriented within the tube wall 116 such that they align with the guidewires 118. As shown in FIGS. 3A and 3B, the guidewires extend from the distal end of the stent 108 and are positioned within the guidetube 110 channels 120. As shown in FIG. 2B, when the guidetube 110 is fully positioned within the stent 102, the guidewires extend through the channels 120 and the guidetube 110 is positioned at the distal end 104 of the stent 102. In some embodiments, there is a slit or notch 148 on the outer surface 112 of the guidetube, along the path of the guidewire 118 so that it can advance to the distal end 106 of the stent 102. The guidewires 118, attached to the inner surface of the stent 102, and the channels 120 are configured to precisely interlock such that the position and orientation of the guidetube 110 when fully inserted into the stent 102 is known.

According to one embodiment, one ore more of the guidewires 118, may be coded, such as with a color coding, e.g., red 118a, green 118b, and/or blue 118c, at the end of the guidewires (not shown). One or more of the channels 120, is also coded, e.g., red, green, and/or blue, at the guidetube 110 proximal end to correspond to the coding on the guidewires. Although color coding is provided as an example of the coding system, other coding systems may be used, such as notches, lines, dots, or other patterns which differentiate the channels so the guide tube is placed in the correct orientation with respect to the stent, as will be understood by those of skill in the art. The coding is used to define the orientation of the guide tube before it is placed. The outer surface of the sleeve may have markers every 10, 15, or 30 degrees so that it can be placed in the correct orientation. The guide tube and the stent must be assembled precisely in term of orientation so that the marker(s) on the sleeve match the marker(s) on the guidewire attached to the distal end of the stent.

Referring again to FIGS. 5A-5C, the guidetube 110 has an outer wall 112, an inner lumen 114 and a tube wall 116 disposed therebetween. The guidetube 110 comprises an elongated cylinder. The outer wall 112 of the guidetube 110 is formed from a material compatible for insertion into a body lumen, such as the esophagus and trachea. Preferably, the guidetube 110 is flexible such that the guidetube 110 is capable of navigating the body lumen. Suitable materials include various polymeric materials suitable for insertion into a body lumen, such as PTFE and FEP, for example.

Preferably, the guidetube 110 has a length of from about 60 cm to about 100 cm, but will vary depending on the size of the patient and position in the body into which the guidetube is placed.

The inner lumen 114 has a shape and internal diameter capable of interlock with the nested cannula. Although the inner lumen 114 is shown as having a hexagonal shape, other shapes are possible such that the nested cannula interlocks with the guidetube inner lumen 114. Preferably, the inner lumen 114 is a polygonal shape, more preferably, the polygonal shape is a 6, 7, or 8 sided polygonal shape. More preferably, the inner lumen is hexagonal. It has been found that the hexagonal shape is a stable platform for interlock and insertability of a nested cannula device.

The channels 120 may be formed from the same or different material as the guidetube 110. Preferably, the channels 120 are positioned equilaterally within the tube wall 116, more preferably, 120 degrees apart. According to one embodiment, the guidewires 118 and channels 120 are individually marked for identification and alignment. Optionally, a lubricous coating is disposed within the channels 120 to facilitate insertion of the guidewires 118 into the channels 120. Although three channels and three guidewires are shown in the accompanying Figures, other embodiments are possible, such as four to six channels and corresponding guidewires.

The stent 102 is removably attachable to the guidetube 110. Preferably, guidetube 110 interlocks with the distal end 106 of the stent 102 such that the stent 102 and guidetube 110 maintain a fixed position with respect to each other after interlock. The plurality of guidewires 118 are threaded through the channels 120 at the guidetube distal end 122 which is advanced to the distal end of the stent 102, and exit outwardly from the guidetube proximal end 124.

In a preferred embodiment, the launcher 100 is introduced into the patient body through the mouth of the patient body and the stent 102 and guidetube 110 are sized for positioning within a patient bronchi. In this embodiment, the guidewires are sized to be positioned within the bronchi and extend externally through the mouth of the patient body. Referring again to FIG. 5A, in some embodiments, the guidetube 110 optionally includes one or more lumens 126 within the tube wall 116, such as three lumens 126 shown in FIG. 5A. The lumens 126 wall are formed for one or more medical procedures within the body, including one or more of: introduction of a medical device, fiber optics, delivery of fluid, and fluid suction.

As shown in FIG. 5B, the lumens 126 may have an entry/exit opening (126a, 126b) at the distal and/or proximate end of the guidetube 110, and/or may have a side entry/exit opening 126c as shown, for example in FIG. 5C.

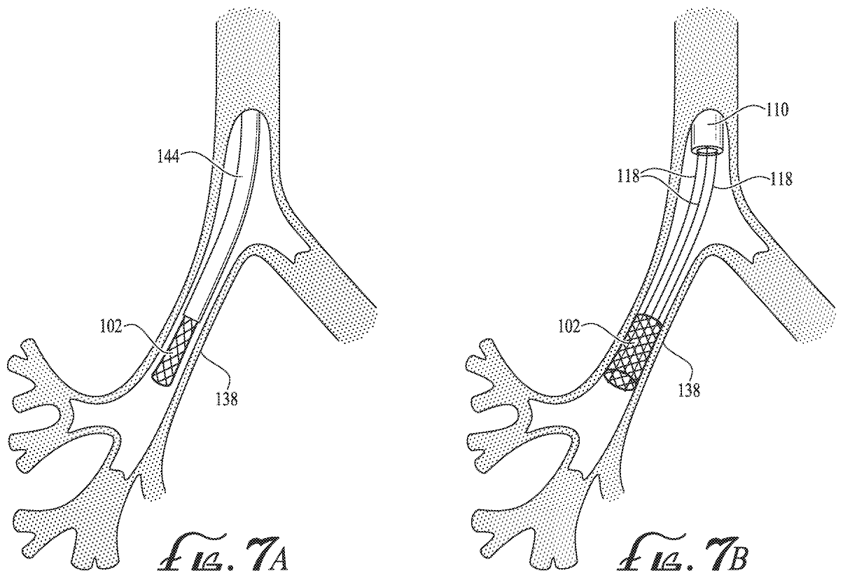

According to another embodiment, an introducer 128 is used for insertion and accesses to the trachea from mouth or nose. Referring now to FIG. 6A, the introducer 128 is a bronchial access assembly, comprising a mouthpiece 130 and a J-tube 132. The J-tube 132 is positionable with the guidetube 110, and may be of the same or different material as the guidetube 110. The J-tube 132, mouthpiece 130, and guidetube 110 may optionally have slits, or other mechanisms, such as notches, to securely fit the guidetube. The mouthpiece 130 comprises a material suitable for insertion into a body, including polymeric materials, such as PEEK and FEP and other materials, such as silicone. Optionally, the mouthpiece and/or J-tube are disposable. In another embodiment, the mouthpiece and J-tube are reusable and made of a material which can be reused and sterilized, such as an autoclavable material, such as materials used for endotracheal tubes, which are rigid at room temperature and are softer once placed in the body. The mouthpiece 130 is rotationable so that the orientation of the guidewire can be controlled to be the same as in the stent. The disk 130 can rotate to fix any misalignment.

According to the present disclosure, the guidetube 110, is positionable on the introducer 132, and the introducer 132 is rotatable to fix the orientation to be the same as the stent 102, and the stent and guidetube 110 interlock. Depending on the patient size, an appropriately sized stent is first selected, depending on the patient size and position within the bronchus. An appropriately sized guidetube 110 is then selected such that it will interlock with the stent 102. Then, an appropriately sized J-tube is selected such that the J-tube is positionable in the trachea and a mouthpiece is selected to fit the J-tube.

To place the stent 102 into the bronchi, a bronchoscope is inserted and the stent 102 is navigated past the trachea to the desired area of the bronchi. A particular problem encountered in bronchoscope applications is that the bronchoscope typically has a relatively large tube diameter and can only turn or be otherwise navigated at the tip. Bronchoscopes are typically large in size and have limited ability to navigate the complex airways of the lung. In addition, lung tissue is particularly fragile, when compared to arterial tissue, and subject to damage and tears when a device is introduced into the bronchia. The introducer 128 facilitates insertion of devices past the bend in the patient's throat and vocal cords, and into the patient's upper trachea.

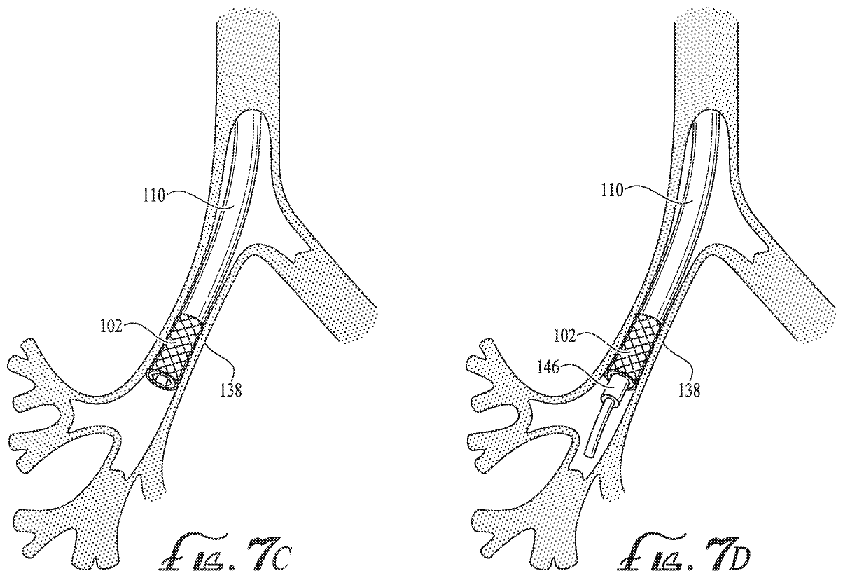

According to one method, as illustrated in FIGS. 6B and 7A-7D, first a bronchial access assembly (e.g., introducer 128) comprising a mouthpiece 130 and a J-tube 132 is inserted into a patient's mouth. FIGS. 6B and 7A-7D are not drawn to scale to illustrate the present disclosure. The mouthpiece 130 is first inserted, followed by the J-tube 132. The J-tube 132 is inserted to extend past the bend in the patient's throat and into a position in the patient's upper trachia, as shown in FIG. 6B. Then, a bronchoscope 134 (not shown) is inserted into the patient's trachea 136, through the mouthpiece 130 and J-tube 132. The stent 102 is inserted though the hollow bore of the mouthpiece 130 and J-tube 132, parallel with the bronchoscope 134 into the left lung 140 of the patient's left bronchus 138. The stent 102 may be inserted within the patient's bronchi through a sleeve 144 as shown in FIG. 7A. The sleeve 144 is retracted, allowing the stent 102 to expand into the desired position within the patient's body. The plurality of guidewires 118 extend to a position outside the patient's body. Preferably, the stent 102 is inserted in a predetermined position within a patient's bronchi, preferably past the corina. Preferably, the coordinate of the stent 102 within the body is verified with the bronchoscope for proper placement.

As shown in FIG. 7B, after insertion of the stent 102, the sleeve 144 is removed, and the guidewires 118 are allowed to protrude outside the patient's body, as described herein. At the mouth of the patient (or other external access point), the guidewires 118 are inserted into corresponding channels 120 on the distal end 122 of the guidetube 110. The guidetube 110 is directed along the guidewires 118 within the bronchi, as shown in FIG. 7B. The guidetube 110 is further directed to a position within the stent 102 until the guidetube 118 interlocks with the stent distal end 104 within the patient bronchi, as shown in FIG. 6C. The nested cannula 146 is inserted through the inner lumen 114 of the guidetube 110 and into the patient's bronchi, in an unlaunched configuration. The stent 102 is used as a launch point to launch the nested cannula 146 into the patient bronchi. Although the above described method is directed to insertion into a patient's bronchi, the launcher 100 may be inserted into other portions of the patient lung, or other body lumens, as will be understood by those of skill in the art.

Preferably, nested cannula 146 is inserted into the patient's bronchi in a predetermined position and the nested cannula interlocks with the guidetube 110. The nested tubes are configured and dimensioned to reach a target location by generating a tube pathway through a set of arcs resulting from a three-dimensional image of a particular anatomical region. Systems and methods describing the nested cannula are described, for example, in U.S. Pat. Nos.: 8,535,336; 9,387,047; 9,895,163; 2011/0201887; and 2014/0371532 and PCT App. Nos. WO 2010/076674; and WO/2010113053.

According to these systems and methods, an image is obtained using a three-dimensional imaging system. The nested tubes (i.e., nested cannula) are configured and dimensioned to reach relatively small and/or complex target locations within a particular anatomical region, and may be formed from a material exhibiting desirable levels of flexibility/elasticity, including a Nitinol material. Each section of the nested tube has a pre-set interlocking shape which interlock to limit rotation of the tubes. The interlocking shapes of the tubes may be the same, or different. Examples of the interlocking shapes include a polygonal interlocking shape, a non-circular closed curve interlocking shape, a polygonal-closed curve hybrid interlocking shape and a keyway interlocking shape. Preferably, the nested tubes have a polygonal interlocking shape. The nested tubes are formed with a variety of shapes and sized, depending on the particular dimensions required for access to a target location in the patient body. The nested tubes, each, individually, may have an outer diameter from about 5 mm down to around 0.2 mm, according to the disclosure.

The three-dimensional imaging system used to target the launch location of the nested cannula and configure and dimension the tubes can be a CT, Ultrasound, PET, SPECT or MRI, and other imaging systems. Typically, the image of the particular anatomical region is used to configure and dimension each of the plurality of tubes to define a particular shape and extension length for each of the plurality of tubes. The defined shape and extension length of each of the plurality of tubes determines whether a target location is reachable. The plurality of tubes may be configured and dimensioned to pre-set shapes and extension lengths for a particular anatomical region. The pre-set plurality of tubes can include alternating curved and straight tubes. Typically, the nested tubes include two or more tubes of a pre-designed curvature, each tube individually is a tube of fixed curvature in order to maintain consistent force on surrounding tubes as they are inserted, which provides a stable shape.

Although the present disclosure has been discussed in considerable detail with reference to certain preferred embodiments, other embodiments are possible. Therefore, the scope of the appended claims should not be limited to the description of preferred embodiments contained herein.

What is claimed is:

1. A launcher for introducing a medical device into a patient body, the launcher comprising:
a stent comprising a hollow body, a proximal end, and a distal end;
a plurality of guidewires affixed at the distal end of the stent and extending from the distal end of the stent to an access point external to the patient body; and
a guidetube comprising:
an elongated tube having an outer wall, an inner lumen and a tube wall disposed therebetween, the guidetube having a shape and diameter capable of insertion into the hollow body of the stent and interlocking with the distal end of the stent, the guidetube extending from the distal end of the stent to the access point external to the patient body; and
a plurality of channels disposed within the tube wall of the guidetube, where the plurality of channels are oriented within the tube wall to align with the guidewires affixed to the stent, and where the guidewires are capable of being removably positioned within the plurality of channels.

2. The launcher according to claim 1, wherein the launcher is introduced into the patient body through the mouth of the patient body and the stent is sized for positioning within a patient bronchi.

3. The launcher according to claim 2, wherein and the plurality of guidewires extend from the distal end of the stent, positioned within the bronchi, and externally to the mouth of the patient body.

4. The launcher according to claim 1, wherein the guidewires and plurality of channels disposed within the guidetube are individually marked for identification.

5. The launcher according to claim 1, wherein the guidetube further comprises one or more lumens within the tube wall.

6. The launcher according to claim 4, wherein the one or more lumens within the tube wall are formed for one or more medical procedures within the body selected from the group consisting of: introduction of a medical device, fiber optics, delivery of fluid, and fluid suction.

7. The launcher according to claim 1 further comprising a bronchial access assembly, the bronchial access assembly comprising a mouthpiece and a J-tube, where the mouthpiece is rotationable to align with the orientation of the guidewires.

8. A stent for introduction of a medical device into a patient body, the stent comprising:
(a) a stent having a proximal end, a distal end, and a continuous lumen between the proximal end and the distal end, said stent having a length and an outside diameter sized for a body passage; and
(b) a plurality of guidewires affixed to the distal end of the stent, the guidewires extending from the distal end of the stent to a position outside the patient body.

9. The stent according to claim 8 further comprising one or more connection members for attachment of the guidewires to the stent.

10. The stent according to claim 8, wherein the stent comprises a shape memory alloy or stainless steel.

11. The stent according to claim 8, wherein the plurality of guidewires comprise a shape memory alloy or stainless steel.

12. The stent according to claim 8, wherein the plurality of guidewires comprise the same material as the stent.

13. The stent according to claim 8, wherein the guidewires comprise a material which is a different material than the stent.

14. The stent according to claim 8, wherein the plurality of guidewires comprise three guidewires.

15. A method for introducing the launcher according to claim 1 into a patient body, the method comprising:
(a) introducing a stent into a patient body, the stent having a hollow body and plurality of guidewires affixed to the distal end of the stent;
(b) positioning the stent within the patient body, the hollow body of the stent being expanded within the patient body, and the plurality of guidewires extending from the distal end of the stent to a position outside the patient body;
(c) providing a guidetube having a tube wall, and a plurality of channels disposed within the tube wall;
(d) inserting the plurality of guidewires, each individually, into one of the channels disposed within the tube wall of the guidetube;
(e) progressing the guidetube along the path provided by the plurality of guidewires into the patient body; and
(f) positioning the guidetube within the hollow cylindrical body of the stent, the guidetube extending from the distal end of the stent to a position beyond the external access point of the patient body.

16. The method according to claim 15, wherein launcher is introduced into a patent bronchi, and the method further comprises inserting a bronchial access assembly into the mouth of the patient body, where the bronchial access assembly comprises a mouthpiece and a J-tube; and extending the J-tube to a position in the upper trachea of the patient body.

17. The method according to claim 15, wherein the stent is positioned in a fixed position which is a predetermined position within the patient's bronchi.

18. A method of introducing a nested cannula into a patient body using the launcher according to claim 1, the method comprising:
(a) determining a predetermined position for positioning a nested cannula within a patient body;
(b) providing a launcher according to claim 1;
(c) positioning the stent within a patient body, the position of the stent being determined by the predetermined position of the nested cannula;
(d) inserting the plurality of guidewires, each individually, into one or more of the channels disposed within the tube wall of the guidetube;
(e) progressing the guidetube along the path provided by the plurality of guidewires into the patient body;
(f) positioning the guidetube within the hollow body of the stent, the guidetube extending from the distal end of the stent to an external access point of the patient body
(g) providing a nested cannula for introduction into the patient body;
(g) inserting the nested cannula through the tube wall of the guidetube; and
(f) positioning the nested cannula within the patient body.

19. The method according to claim 18, wherein the nested cannula is inserted into a patient's bronchi in a predetermined position.

20. The method according to claim 18, wherein the nested cannula interlocks with the guidetube.

21. The method according to claim 18, wherein the method further comprises inserting a bronchial access assembly into a patients mouth, the bronchial access assembly comprising a mouthpiece and a J-tube, and extending the J-tube to a position in the patient's upper trachea.
